(19) United States
(12) Patent Application Publication
(10) Pub. No.: US 2008/0015190 A1
(43) Pub. Date: Jan. 17, 2008

(54) Inhibitors of Histone Deacetylase

(76) Inventors: Prasun K. Chakravarty, Edison, NJ (US); Howard Kuo, South Plainfield, NJ (US); Jay M. Matthews, Lansdale, PA (US); Peter T. Meinke, Cranford, NJ (US)

Correspondence Address: MERCK AND CO., INC, P O BOX 2000, RAHWAY, NJ, 07065-0907, US

(21) Appl. No.: 11/629,588

(22) PCT Filed: Jul. 8, 2005
(86) PCT No.: PCT/US05/24512

§ 371 Date: Dec. 14, 2006

Related U.S. Application Data
(60) Provisional application No. 60/587,233, filed on Jul. 12, 2004.

(51) Int. Cl. A61K 31/16; A61K 31/4184; A61K 31/42; A61K 31/428; A61K 31/445; A61K 31/47; A61K 31/5375; A61K 31/5377; A61P 25/00; A61P 35/00; C07C 259/10; C07D 211/06; C07D 215/36; C07D 261/10; C07D 277/60; C07D 295/14; C07D 413/04; C07D 487/00

(52) U.S. Cl. 514/235.2; 514/237.5; 514/311; 514/313; 514/330; 514/367; 514/380; 514/395; 514/575; 544/128; 544/160; 546/177; 546/225; 548/171; 548/243; 548/306.4; 562/622

(57) ABSTRACT

The present invention relates to hydroxamic acid derivatives that are inhibitors of histone deacetylase (HDAC). The compounds of the present invention are useful for treating cellular proliferative diseases, including cancer. Further, the compounds of the present invention are useful for treating neurodegenerative diseases, schizophrenia and stroke among other diseases. Further, the compounds of the present invention have antiprotozoal properties.

### BACKGROUND OF THE INVENTION

DNA in the nucleus of the cell exists as a hierarchy of compacted chromatin structures. The basic repeating unit in chromatin is the nucleosome. The nucleosome consists of a histone octamer of proteins in the nucleus of the cell around which DNA is wrapped twice. The orderly packaging of DNA in the nucleus plays an important role in the functional aspects of gene regulation. Covalent modifications of the histones have a key role in altering chromatin higher order structure and function and ultimately gene expression. The covalent modification of histones, such as acetylation, occurs by enzymatically mediated processes.

Regulation of gene expression through the inhibition of the nuclear enzyme histone deacetylase (HDAC) is one of several possible regulatory mechanisms whereby chromatin activity can be affected. The dynamic homeostasis of the nuclear acetylation of histones can be regulated by the opposing activity of the enzymes histone acetyl transferase (HAT) and histone deacetylase (HDAC). Transcriptionally silent chromatin can be characterized by nucleosomes with low levels of acetylated histones. Acetylation reduces the positive charge of histones, thereby expanding the structure of the nucleosome and facilitating the interaction of transcription factors with the DNA. Removal of the acetyl group restores the positive charge, condensing the structure of the nucleosome. Histone acetylation can activate DNA transcription, enhancing gene expression. Histone deacetylase can reverse the process and can serve to repress gene expression. See, for example, Grunstein, Nature 389, 349-352 (1997); Pazin et al., Cell 89, 325-328 (1997); Wade et al., Trends Biochem. Sci. 22, 128-132 (1997); and Wolffe, Science 272, 371-372 (1996).

### SUMMARY OF THE INVENTION

The present invention relates to hydroxamic acid derivatives that are inhibitors of histone deacetylase (HDAC). The compounds of the present invention are useful for treating cellular proliferative diseases, including cancer. The compounds of the present invention are also useful for treating neurodegenerative diseases, schizophrenia and stroke among other diseases. Further, the compounds of the present invention have antiprotozoal properties.

### DETAILED DESCRIPTION OF THE INVENTION

The compounds of this invention are useful in the inhibition of histone deacetylase. A first embodiment of the instant invention is a compound as illustrated by Formula I:

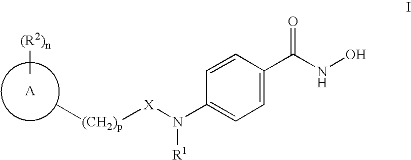

wherein:

a is 0 or 1; b is 0 or 1; m is 0, 1 or 2; n is 0, 1, 2, 3, 4 or 5; and p is 0, 1, 2 or 3;

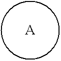

is cycloalkyl, aryl, heterocyclyl or

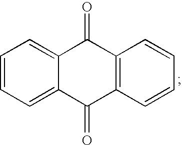

X is C═O or S(O)₂;

R¹ is selected from: H and (C₁-C₆)alkyl;

R² is independently selected from: oxo, OH, (C═O)ₐO_{b}(C₂-C₁₀)alkenyl, (C═O)ₐO_{b}(C₂-C₁₀)alkynyl, NO₂, (C═O)ₐO_{b}(C₁-C₆)alkyl, CN, (C═O)ₐO_{b}(C₃-C₁₀)cycloalkyl, halogen, (C═O)ₐ—N(R^{a})₂, CF₃, OH, NH—S(O)ₘ—R^{a}, (C═O)ₐO_{b}-heterocyclyl, (C═O)ₐO_{b}-aryl, S(O)ₘ—R^{a}, NH(C═O)R^{a}, N═N-aryl-N(R^{a})₂, (C₁-C₆)alkyl-aryl and heterocyclyl, said alkyl, alkenyl, alkynyl, cycloalkyl, aryl and heterocyclyl optionally substituted with one to three R^{b};

R^{a} is independently selected from: H and (C₁-C₆)alkyl;

R^{b} is independently selected from: oxo, NO₂, N(R^{a})₂, OH, CN, halogen, CF₃ and (C₁-C₆)alkyl;

or a pharmaceutically acceptable salt or stereoisomer thereof.

A second embodiment of the instant invention is a compound as illustrated by Formula I;

wherein:

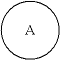

is phenyl, heterocyclyl or

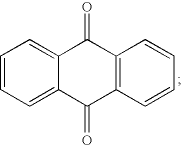

p is 0 or 1;

R¹ is CH₃;

and all substituents and variables are as defined in the first embodiment;

or a pharmaceutically acceptable salt or stereoisomer thereof.

A third embodiment of the instant invention is a compound as illustrated by Formula I;

wherein:

R² is independently selected from: NO², (C═O)ₐO_{b}(C₁-C₆)alkyl, CN, (C₃-C₁₀)cycloalkyl, halogen, (C═O)ₐ—N(R^{a})₂, CF₃, OH, NH—S(O)ₘ—R^{a}, (C═O)ₐ-heterocyclyl, (C═O)ₐ-aryl, S(O)ₘ—R^{a}, NH(C═O)R^{a}, N═N-aryl-N(R^{a})₂, (C₁-C₆)alkyl-aryl and heterocyclyl, said alkyl, cycloalkyl, aryl and heterocyclyl optionally substituted with one to three R^{b};

R^{a} is independently selected from: H and (C₁-C₆)alkyl;

R^{b} is independently selected from: halogen, CF₃ and (C₁-C₆)alkyl;

and all substituents and variables are as defined in the second embodiment;

or a pharmaceutically acceptable salt or stereoisomer thereof.

The compounds of the present invention may have asymmetric centers, chiral axes, and chiral planes (as described in: E. L. Eliel and S. H. Wilen, Stereochemistiy of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention. In addition, the compounds disclosed herein may exist as tautomers and both tautomeric forms are intended to be encompassed by the scope of the invention, even though only one tautomeric structure is depicted.

When any variable (e.g. R¹ and R², etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on. The term “cycloalkyl” means a monocyclic, bicyclic or polycyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on. In an embodiment of the invention the term “cycloalkyl” includes the groups described immediately above and further includes monocyclic unsaturated aliphatic hydrocarbon groups. For example, “cycloalkyl” as defined in this embodiment includes cyclopropyl, methyl-cyclopropyl, 2,2-dimethyl-cyclobutyl, 2-ethyl-cyclopentyl, cyclohexyl, cyclopentenyl, cyclobutenyl, 7,7-dimethylbicyclo[2.2.1]heptyl and so on.

The term “alkylene” means a hydrocarbon diradical group having the specified number of carbon atoms. For example, “alkylene” includes —CH₂—, —CH₂CH₂— and the like.

“Alkoxy” represents either a cyclic or non-cyclic alkyl group of indicated number of carbon atoms attached through an oxygen bridge. “Alkoxy” therefore encompasses the definitions of alkyl and cycloalkyl above.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, —CH(CH₃)CH₂CH(CH₃)Ph, and so on.

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

The term “heterocycle” or “heterocyclyl” as used herein is intended to mean a 4- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrahydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrothiopyranyl, tetrahydroisoquinolinyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

As appreciated by those of skill in the art, “halo” or “halogen” as used herein is intended to include chloro (Cl), fluoro (F), bromo (Br) and iodo (I).

In an embodiment,

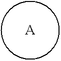

is: phenyl, heterocyclyl, or

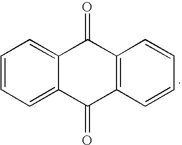

In an embodiment, p is 0 or 1.

In another embodiment, p is 0.

In an embodiment, X is C═O.

In another embodiment, X is S(O)₂.

In an embodiment, R¹ is H.

In another embodiment, R¹ is CH₃.

In an embodiment, R² is independently selected from: oxo, OH, (C═O)ₐO_{b}(C₂-C₁₀)alkenyl, (C═O)ₐO_{b}(C₂-C₁₀)alkynyl, NO₂, (C═O)ₐO_{b}(C₁-C₆)alkyl, CN, (C═O)ₐO_{b}(C₃-C₁₀)cycloalkyl, halogen, (C═O)ₐ—N(R^{a})₂, CF₃, OH, NH—S(O)ₘ—R^{a}, (C═O)ₐO_{b}-heterocyclyl, (C═O)ₐO_{b}-aryl, S(O)ₘ—R^{a}, NH(C═O)R^{a}, N═N-aryl-N(R^{a})₂, (C₁-C₆)alkyl-aryl and heterocyclyl, said alkyl, alkenyl, alkynyl, cycloalkyl, aryl and heterocyclyl optionally substituted with one to three R^{b}.

In another embodiment, R² is independently selected from: NO₂, (C═O)ₐO_{b}(C₁-C₆)alkyl, CN, (C₃-C₁₀)cycloalkyl, halogen, (C═O)ₐ—N(R^{a})₂, CF₃, OH, NH—S(O)ₘ—R^{a}, (C═O)ₐ-heterocyclyl, (C═O)ₐ-aryl, S(O)ₘ—R^{a}, NH(C═O)R^{a}, N═N-aryl-N(R^{a})₂, (C₁-C₆)alkyl-aryl and heterocyclyl, said alkyl, cycloalkyl, aryl and heterocyclyl optionally substituted with one to three R^{b}.

In yet another embodiment, when R² is aryl, said aryl is phenyl.

In yet another embodiment, when R² is heterocyclyl, said heterocyclyl is selected from:

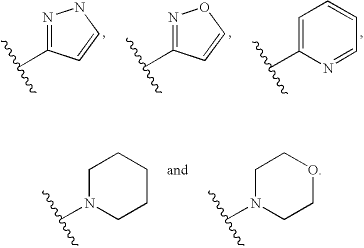

In an embodiment, R^{b} is independently selected from: oxo, NO₂, N(R^{a})₂, OH, CN, halogen, CF₃ and (C₁-C₆)alkyl.

In another embodiment, R^{b} is independently selected from: halogen, CF₃ and (C₁-C₆)alkyl.

Included in the instant invention is the free form of compounds of Formula I, as well as the pharmaceutically acceptable salts and stereoisomers thereof. Some of the specific compounds exemplified herein are the protonated salts of amine compounds. The term “free form” refers to the amine compounds in non-salt form. The encompassed pharmaceutically acceptable salts not only include the salts exemplified for the specific compounds described herein, but also all the typical pharmaceutically acceptable salts of the free form of compounds of Formula I. The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant compounds can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the compounds of this invention include the conventional non-toxic salts of the compounds of this invention as formed by reacting a basic instant compound with an inorganic or organic acid. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The preparation of the pharmaceutically acceptable salts described above and other typical pharmaceutically acceptable salts is more fully described by Berg et al., “Pharmaceutical Salts,” J. Pharm. Sci., 1977:66:1-19.

It will also be noted that the compounds of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

The compounds of this invention may be prepared by employing reactions as shown in the following schemes, in addition to other standard manipulations that are known in the literature or exemplified in the experimental procedures. The illustrative schemes below, therefore, are not limited by the compounds listed or by any particular substituents employed for illustrative purposes. Substituent numbering as shown in the schemes does not necessarily correlate to that used in the claims and often, for clarity, a single substituent is shown attached to the compound where multiple substituents are allowed under the definitions of Formula I hereinabove

### Reaction Schemes

As shown in Schemes 1, the sulfonamide compounds of this invention can readily be prepared from an appropriate p-aminobenzoic acid derivative 1, using the general chemistry outlined. These p-aminobenzoic acid derivatives can be either purchased from commercial sources or prepared by those skilled in the art using standard chemistry.

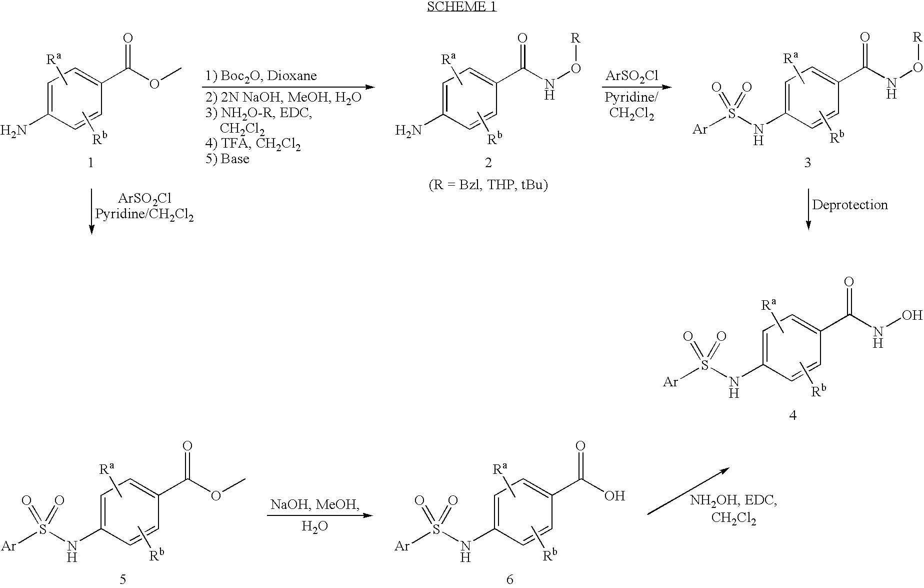

In one such approach, as shown above, the derivative 1 can be N-protected with a suitable protecting group reacted, such as Boc₂O, (suitable protecting groups are described in Protecting Groups in Organic Synthesis, 3rd Edition, Greene, T. W. and Wuts, P. G. M.; Wiley Interscience, 1999 and Kocienski, P. J. Protecting Groups, Thieme, 1994) followed by base hydrolysis to provide the corresponding carboxylic acid, which can then be reacted with an appropriate O-protected (such as with either benzyl, t-butyl, THP or t-butyldimethylsilyl) hydroxylamine derivative in the presence of a coupling agent, such as DCC or EDC to form the corresponding hydroxamate. Methods for coupling of carboxylic acids (and acid derivatives) with amino component to form carboxamides are well known in the art, suitable methods are described, for example, in March, J. Advanced Organic Chemistry, 3rd edition, John Wiley & Sons, 1985, pp. 370-376. The N-protecting group can be then removed under acidic condition using strong acid, such as trifluoroacetic acid, to provide the hydroxamic acid derivative 2, which can be reacted with an appropriate sulfonyl chloride in the presence of a suitable base to give the sulfonamide 3. Finally, the O-protecting group can be removed to yield the titled compounds 4 of this invention. Alternatively, 1 can be reacted with an appropriate sulfonyl chloride to form the sulfonamide derivative 5. The carboxylic acid derivative 6 (obtained from 5) then can be converted into 4 as outlined.

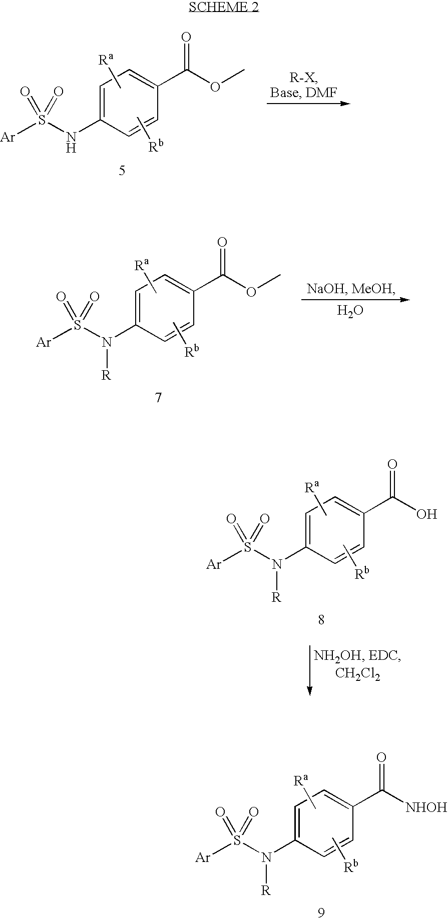

The sulfonamide 5 can be reacted with an appropriate alkylating agent in the presence of a suitable base, as outlined in Scheme 2, to provide the ester 7. The acid 8, obtained from the ester 7, can be converted into the desired hydroxamate 9. In some cases, further synthetic manipulation on the complete molecule can lead to other analogues. The aromatic sulfonyl chlorides can be obtained either from commercial sources or can be readily prepared using various methods available to one skilled in the art [as described in Org. Proc. Res. Development (2003), 7(6), 921-924; Tett. Lett. (2003), 44(21), 4153-4256; J. Org. Chem. (2003), 68(14), 5525-5573; Bioorg. Med. Chem. (2002), 10(11), 3529-3544; Tetrahedron (2003), 59(8), 1317-1325; J. Heterocyclic Chem. (2002), 39(5), 1055-1059; J. Med. Chem. (2002), 45(5), 1086-1097].

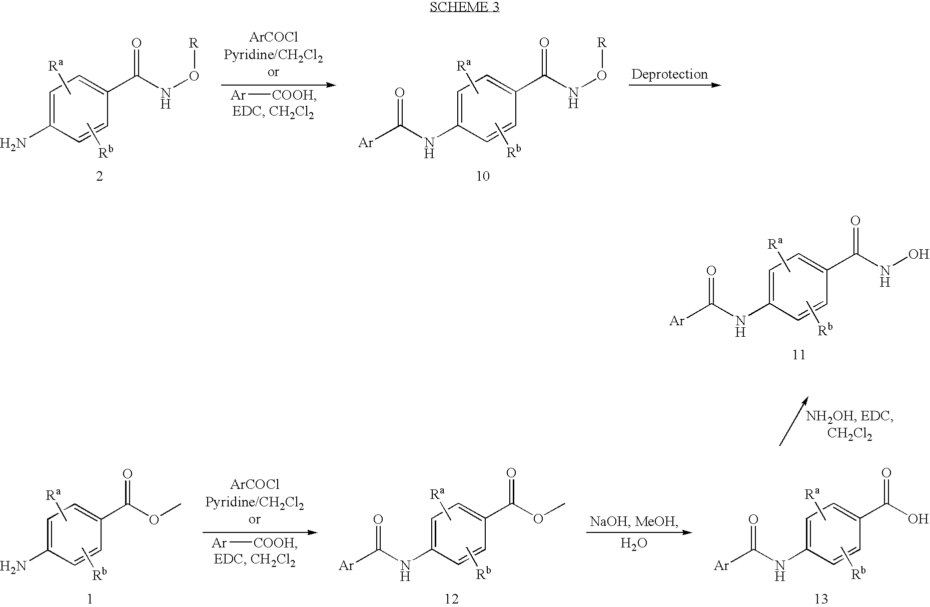

As shown in Scheme 3, the amide compounds of this invention can be synthesized by reacting 2 with an appropriate acid chloride or a carboxylic acid as outlined to give 10, which upon removal of the protecting group can provide the titled hydroxamate 11. The hydroxamate 11 can also be prepared from 1 as outlined above in Scheme 3. The corresponding N-susbstituted amide compounds 16 can be prepared from 12, as outlined in Scheme 4.

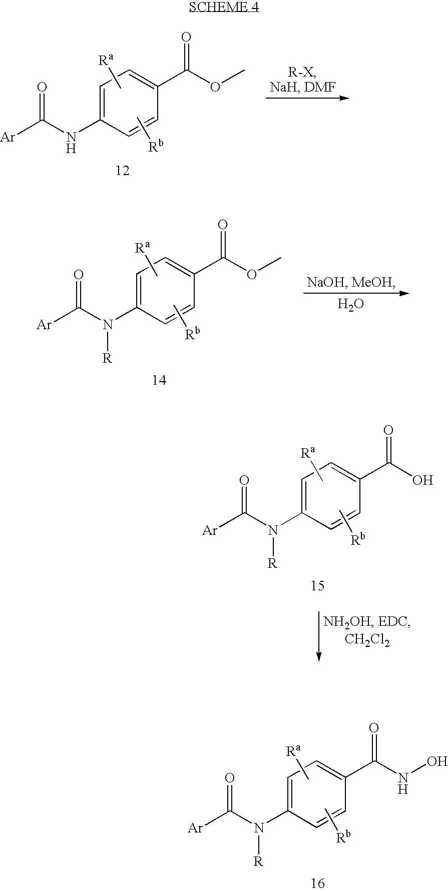

### Utility

The compounds of the invention find use in a variety of applications. The compounds of the invention are histone deacetylase (HDAC) inhibitors useful in the treatment of cancer among other diseases. HDACs catalyse the removal of acetyl groups from lysine residues on proteins, including histones and HDAC inhibitors show diverse biological functions including affecting gene expression, cell differentiation, cell cycle progression, growth arrest, and/or apoptosis. See J. Med. Chem. 2003, 46:5097 and Curr. Med. Chem. 2003, 10:2343.

The compounds of the invention are used to treat cellular proliferation diseases. Disease states which can be treated by the methods and compositions provided herein include, but are not limited to, cancer (further discussed below), neurodegenerative diseases, schizophrenia and stroke

The compounds, compositions and methods provided herein are particularly deemed useful for the treatment of cancer including solid tumors such as skin, breast, brain, cervical carcinomas, testicular carcinomas, etc. In particular, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

The compounds of the invention are also useful in preparing a medicament that is useful in treating the cellular proliferation diseases above, in particular cancer.

The compounds of the instant invention may also be useful in the treatment or prevention of neurodegenerative diseases, including, but not limited to, polyglutamine-expansion-related neurodegeneration, Huntington's disease, Kennedy's disease, spinocerebellar ataxia, dentatorubral-pallidoluysian atrophy (DRPLA), protein-aggregation-related neurodegeneration, Machado-Joseph's disease, Alzheimer's disease, Parkinson's disease, amyotrophic lateral sclerosis, spongiform encephalopathy, a prion-related disease and multiple sclerosis (MS). See WO 02/090534 and WO 03/083067.

The compounds of the invention are also useful in preparing a medicament that is useful in treating or preventing neurodegenerative diseses.

The compounds of the invention may also be useful in the treatment or prevention of schizophrenia. See WO 02/090534.

The compounds of the invention are also useful in preparing a medicament that is useful in treating or preventing schizophrenia.

The compounds of the invention may also be useful in the treatment or prevention of inflammatory diseases, including, but not limited to stroke. Leoni et al., PNAS, 99(5):2995-3000 (2002) and Suuronen et al., J. Neurochem. 87:407-416 (2003).

The compounds of the invention are also useful in preparing a medicament that is useful in treating or preventing inflammatory diseases such as stroke.

The compounds of the present invention are also useful in the inhibition of smooth muscle cell proliferation and/or migration and are thus useful in the prevention and/or treatment of restenosis, for example after angioplasty and/or stent implantation.

The compounds of the invention are also useful in preparing a medicament that is useful in treating or preventing restenosis.

In one embodiment, smooth muscle cell proliferation and/or migration is inhibited and restenosis is prevented and/or treated by providing a stent device having one or more of the compounds of the instant invention in or on the stent device, e.g. coated onto the stent device. The stent device is designed to controllably release the compounds of the invention, thereby inhibiting smooth miscle cell proliferation and/or migration and preventing and/or treating restenosis.

Stenosis and restenosis are conditions associated with a narrowing of blood vessels. Stenosis of blood vessels generally occurs gradually over time. Restenosis, in contrast, relates to a narrowing of blood vessels following an endovascular procedure, such as balloon angioplasty and/or stent implantation, or a vascular injury.

Balloon angioplasty is typically performed to open a stenotic blood vessel; stenting is usually performed to maintain the patency of a blood vessel after, or in combination with, balloon angioplasty. A stenotic blood vessel is opened with balloon angioplasty by navigating a balloon-tipped catheter to the site of stenosis, and expanding the balloon tip effectively to dilate the occluded blood vessel. In an effort to maintain the patency of the dilated blood vessel, a stent may be implanted in the blood vessel to provide intravascular support to the opened section of the blood vessel, thereby limiting the extent to which the blood vessel will return to its occluded state after release of the balloon catheter. Restenosis is typically caused by trauma inflicted during angioplasty, effected by, for example, balloon dilation, atherectomy or laser ablation treatment of the artery. For these procedures, restenosis occurs at a rate of about 30% to about 60% depending on the vessel location, lesion length and a number of other variables. This reduces the overall success of the relatively non-invasive balloon angioplasty and stenting procedures

Restenosis is attributed to many factors, including proliferation of smooth muscle cells (SMC). SMC proliferation is triggered by the initial mechanical injury to the intima that is sustained at the time of balloon angioplasty and stent implantation. The process is characterized by early platelet activation and thrombus formation, followed by SMC recruitment and migration, and, finally, cellular proliferation and extracellular matrix accumulation. Damaged endothelial cells, SMCs, platelets, and macrophages secrete cytokines and growth factors which promote restenosis. SMC proliferation represents the final common pathway leading to neointimal hyperplasia. Therefore, anti-proliferative therapies aimed at inhibiting specific regulatory events in the cell cycle may constitute the most reasonable approach to restenosis after angioplasty.

In another aspect the present invention provides a method for the treatment of protozoal infections comprising administering to a host suffering from a protozoal infection a therapeutically effective amount of a compound according to formula (I) which inhibits histone deacetylase. A therapeutically effective amount is one that is sufficient to inhibit histone deacetylase of the causative protozoa.

The compounds of the invention are also useful in preparing a medicament that is useful in treating or preventing protozoal infections.

Histone deacetylase inhibitors are useful as antiprotozoal agents. As such, the compunds of the present invention can be used in the treatment and prevention of protozoal diseases in human and animals, including poultry. Examples of protozoal diseases against which histone deacetylase inhibitors may be used, and their respective causative pathogens, include: 1) amoebiasis (Dientamoeba sp., Entamoeba histolytica); 2) giardiasis (Giardia lamblia); 3) malaria (Plasmodium species including P. vivax, P. falciparum, P. malariae and P. ovale); 4) leishmaniasis (Leishmania species including L. donovani, L. tropica, L. mexicana, and L. braziliensis); 5) trypanosomiasis and Chagas disease (Trypanosoma species including T. brucei, T. theileri, T. rhodesiense, T. gambiense, T. evansi, T. equiperdum, T. equinum, T. congolense, T. vivax and T. cruzi); 6) toxoplasmosis (Toxoplasma gondii); 7) neosporosis (Neospora caninum); 8) babesiosis (Babesia sp.); 9) cryptosporidiosis (Cryptosporidium sp.); 10) dysentary (Balantidium coli); 11) vaginitis (Trichomonas species including T. vaginitis, and T. foetus); 12) coccidiosis (Eimeria species including E. tenella, E. necatrix, E. acervulina, E. maxima and E. brunetti, E. mitis, E. bovis, E. melagramatis, and Isospora sp.); 13) enterohepatitis (Histomonas gallinarum), and 14) infections caused by Anaplasma sp., Besnoitia sp., Leucocytozoan sp., Microsporidia sp., Sarcocystis sp., Theileria sp., and Pneumocystis carinii.

Histone deacetylase inhibitors of the present invention are preferably used in the treatment or prevention of protozoal infections caused by a member of the sub-phylum Apicomplexans. More preferably histone deacetylase inhibitors are preferably used in the treatment or prevention of malaria, toxoplasmosis, and cryptosporidiosis in humans and animals; and in the management of coccidiosis, particularly in poultry, either to treat coccidial infection or to prevent the occurrence of such infection. Further, although not caused by an Apicomplexan, trypanosomiasis may be treated by histone deacetylase inhibitors.

In the case that a histone deacetylase inhibitor of the present invention is expected to be administered on a chronic basis, such as in the prevention of coccidiosis in poultry, the histone deacetylase inhibitor preferably is selective for protozoal over the host histone deacetylase. Long term administration of such a selective inhibitor would minimize adverse effects to the host due to histone deacetylase inhibition.

The compounds of this invention may be administered to mammals, preferably humans, either alone or in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a human subject, the daily dosage will normally be determined by the prescribing physician with the dosage generally varying according to the age, weight, sex and response of the individual patient, as well as the severity of the patient's symptoms.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

The instant compounds are also useful in combination with known therapeutic agents and anti-cancer agents. For example, instant compounds are useful in combination with known anti-cancer agents. Combinations of the presently disclosed compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The instant compounds are particularly useful when co-administered with radiation therapy.

In an embodiment, the instant compounds are also useful in combination with known anti-cancer agents including the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, and 4-demethoxy-3-deamino-3-aziridinyl-4-methylsulphonyl-daunorubicin (see WO 00/50032).

An example of a hypoxia activatable compound is tirapazamine.

Examples of proteasome inhibitors include but are not limited to lactacystin and bortezomib

Examples of microtubule inhibitors/microtubule-stabilising agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-k1]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1 -de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amnino]-3-hydroxy-7H-indeno[2,1-c]quinolin-7-one, and dimesna.

Examples of inhibitors of mitotic kinesins, and in particular the human mitotic kinesin KSP, are described in PCT Publications WO 01/30768, WO 01/98278, WO 03/050,064, WO 03/050,122, WO 03/049,527, WO 03/049,679, WO 03/049,678 and WO 03/39460 and pending PCT Appl. Nos. US03/06403 (filed Mar. 4, 2003), US03/15861 (filed May 19, 2003), US03/15810 (filed May 19, 2003), US03/18482 (filed Jun. 12, 2003) and US03/18694 (filed Jun. 12, 2003). In an embodiment inhibitors of mitotic kinesins include, but are not limited to inhibitors of KSP, inhibitors of MKLP1, inhibitors of CENP-E, inhibitors of MCAK, inhibitors of Kif14, inhibitors of Mphosph1 and inhibitors of Rab6-KIFL.

“Inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11 -diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp.1394-1401(1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromnb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in PCT Publication WO 03/013,526 and U.S. Ser. No. 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Inhibitors of cell proliferation and survival signaling pathway” refer to pharmaceutical agents that inhibit cell surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in (WO 03/086404, WO 03/086403, WO 03/086394, WO 03/086279, WO 02/083675, WO 02/083139, WO 02/083140 and WO 02/083138), inhibitors of Raf kinase (for example BAY-43-9006 ), inhibitors of MEK (for example CI-1040 and PD-098059) and inhibitors of mTOR (for example Wyeth CCI-779 and Ariad AP23573). Such agents include small molecule inhibitor compounds and antibody antagonists.

“Apoptosis inducing agents” include activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823, U.S. Pat. No. 5,633,272, and U.S. Pat. No. 5,932,598, all of which are hereby incorporated by reference.

Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to: parecoxib, CELEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-k1]pyrrolo[3,4-i][1,6]benzodiazocin-1-one, SH268, genistein, ST571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malingpancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999;274:9116-9121; Invest. Opthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy)phenoxy)propoxy)-2-ethylchromane-2-carboxylic acid (disclosed in U.S. Ser. Nos. 60/235,708 and 60/244,697).

Another embodiment of the instant invention is the use of the presently disclosed compounds in combination with anti-viral agents (such as nucleoside analogs including ganciclovir for the treatment of cancer. See WO 98/04290.

Another embodiment of the instant invention is the use of the presently disclosed compounds in combination with gene therapy for the treatment of cancer. For an overview of genetic strategies to treating cancer see Hall et al (Am J Hum Genet 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p 53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998;5(8): 1105-13), and interferon gamma (J Immunol 2000; 164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In an embodiment, an anti-emesis agent selected from a neurokinin-1 receptor antagonist, a 5HT3 receptor antagonist and a corticosteroid is administered as an adjuvant for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, isoprinosine and Zadaxin.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

Thus, the scope of the instant invention encompasses the use of the instantly claimed compounds in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an anti-viral agent, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an agent that interfers with a cell cycle checkpoint, an apoptosis inducing agent and a bisphosphonate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In an embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF. In an embodiment, the estrogen receptor modulator is tamoxifen or raloxifene.

Also included in the scope of the claims is a method of treating cancer that comprises administering a therapeutically effective amount of a compound of Formula I in combination with radiation therapy and/or in combination with a compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an anti-viral agent, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an agent that interfers with a cell cycle checkpoint, an apoptosis inducing agent and a bisphosphonate.

And yet another embodiment of the invention is a method of treating cancer that comprises administering a therapeutically effective amount of a compound of Formula I in combination with paclitaxel or trastuzumab.

The invention further encompasses a method of treating or preventing cancer that comprises administering a therapeutically effective amount of a compound of Formula I in combination with a COX-2 inhibitor.

The instant invention also includes a pharmaceutical composition useful for treating or preventing cancer that comprises a therapeutically effective amount of a compound of Formula I and a compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an anti-viral agent, an inhibitor of cell proliferation and survival signaling, an agent that interfers with a cell cycle checkpoint, an apoptosis inducing agent and a bisphosphonate.

These and other aspects of the invention will be apparent from the teachings contained herein.

All patents, publications and pending patent applications identified are hereby incorporated by reference.

Abbreviations used in the description of the chemistry and in the Examples that follow are: AcOH (acetic acid); DCE (dichloromethane); DIBAL-H (diisobutylaluminum hydride); DIEA (diisopropylethylamine); DME (ethylene glycol dimethyl ether); DMAP (4,4-Dimethylaminopyridine); DMF (dimethylformamide); DMSO (dimethyl sulfoxide); DTT (dithiothreitol); EDC (ethyl-3(3-dimethylaminopropyl)carbodiimide); EtOAc (ethyl acetate); FACS (fluorescence activated cell sorting); FITC (Fluorescein isothiocyanate); IPTG (Isopropyl-beta-D-thiogalactopyranoside); LDA (lithium diisopropylamide); LHMDS (lithium hexamethyldisilazide); mCPBA (m-chloroperoxybenzoic acid); MS (mass spectrometry); N S (sodium bistrimethylsilylamide); NMR (nuclear magnetic resonance); PMSF (phenylmethylsulphonyl fluoride); PyBop (1H-1,2,3-benzotriazol-1-yloxy)(tripyrrolidin-1-yl)phosphonium hexafluorophosphate); SiO₂ (silica gel); TBAI (tetra-n-butylammonium iodide); TEA (triethyl amine); THF (tetrahydrofuran); TFA (trifluoroacteic acid); TMSCN (trimethylsilylcyanide); and TsCl (p-toluenesulfonyl chloride).

### EXAMPLE 1

### See Compound Number 1

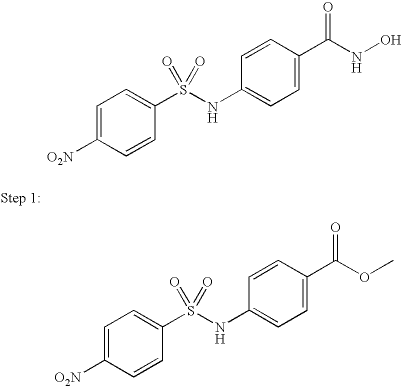

To a solution of p-aminobenzoic acid methyl ester (0.72 g, 4.75 mMol) in CH₂Cl₂ (10 mL) was added N-methyl morpholine (0.079 mL, 7.12 mMol) at 0° C. After stirring for 15 min, p-nitrophenyl sulfonyl chloride (0.96 g, 4.33 mMol) was added at 0° C., stirred for 1 h at that temperature and then at room temperature overnight. The reaction was diluted with CH₂Cl_{2,} washed with 1N HCl and water, then dried over anhydrous MgSO₄. The crude product obtained, after removal of the solvent, was purified by chromatography on silica eluting with 40% EtOAc/petroleum ether to yield the desired sulfonamide. MS(ES) C₁₄H₁₂N₂O₆S requires: 336, found: 337 (M+H⁺).

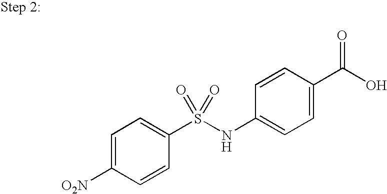

To a solution of the ester from Step 1 (0.08 g, 0.24 mMol) in MeOH (2 mL) was added 2N NaOH (2 mL). After stirring at RT for 3 h, the solvent was removed in vacuo. The residue obtained was dissolved in water (5 mL) and acidified with cold 1N HCl. The precipitate formed was filtered, washed with water and air dried to yield the desired carboxylic acid.

¹H NMR (400 MHz, d6-DMSO) δ 8.34 (d, 2H, J=7.0 Hz), 8.06 (d, 2H, J=7.0 Hz), 7.87 (d, 2H, J=7.0 Hz), 7.2 (d, 2H, J=7.0 Hz), 4.85 (1H, broad s). MS(ES) C₁₃H₁₀N₂O₆S requires: 323, found: 324 (M+H⁺).

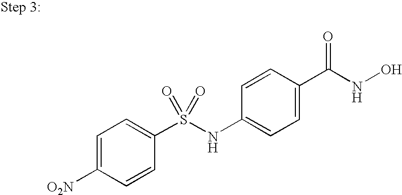

A mixture of the carboxylic acid from Step 2 (0.050 g, 0.155 mMol) and O-t-butyldimethylsilyl (0.057 mL, 0.47 mMol) was dissolved in CH₂Cl₂ (1.5 mL), and cooled to 0° C. EDC (0.0.033 g, 0.172 mMol) was then added, and the mixture was stirred at room temperature for 3 h. The reaction mixture was diluted with CH₂Cl₂, stirred with 2 N HCl for 1 h at room temperature, washed with water, dried (Na₂SO₄) and concentrated under reduced pressure. The residue obtained was dissolved in THF (1 mL) and treated with AcOH overnight. The crude product obtained was purified by column chromatography on silica-gel using with CH₂Cl₂/MeOH (60:1) to yield the titled hydroxamic acid.

¹H NMR (400 MHz, d6-DMSO) δ 8.36 (d, 2H, J=7.0 Hz), 8.16 (d, 2H, J=7.0 Hz), 7.94 (d, 2H, J=7.0 Hz), 7.32 (d, 2H, J=7.0 Hz). MS(ES) C₁₃H₁₁N₃O₆S requires: 337, found: 338 (M+H⁺).

### EXAMPLE 2

### See Compound Number 71

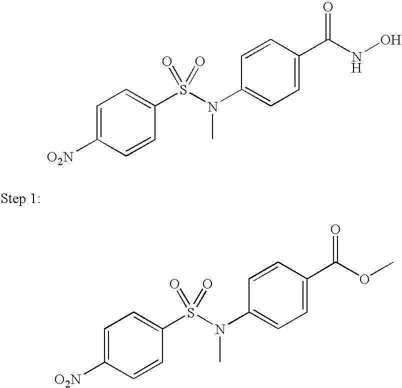

To a solution of the ester compound from Step 1 of Example 1 (0.21 g, 0.62 mMol) in DMF (10 mL) was added Cs₂CO₃ (0.41 g, 2.5 mMol) at room temperature. After stirring for 30 min, MeI (0.16 ml, 2.66 mMol) was added and stirring continued overnight at that temperature. The reaction was diluted with water and extracted with CH₂Cl₂. The organic phase was washed with water, then dried over anhydrous MgSO₄. The crude product obtained, after removal of the solvent, was purified by chromatography on silica eluting with 33% EtOAc/petroleum ether to yield the desired sulfonamide.

¹H NMR (400 MHz, CD₃OD) δ 8.36 (d, 2H, J=7.0 Hz), 8.0 (d, 2H, J=7.0 Hz), 7.8 (d, 2H, J=7.0 Hz), 7.24 (d, 2H, J=7.0 Hz), 3.92 (3H, s). MS(ES) C₁₅H₁₄N₂O₆S requires: 350, found: 351 (M+H⁺).

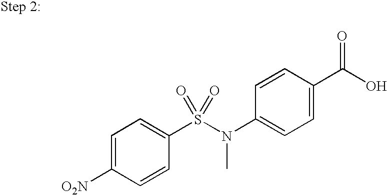

To a solution of the ester from Step 1 above (0.165 g, 0.47 mMol) in a mixture of MeOH (5 mL) and THF (10 mL) was added 2N NaOH (5 mL). The mixture was refluxed for 1 h and then the solvent was removed in vacuo. The residue obtained was dissolved in water (5 mL) and acidified with cold 1N HCl. The precipitate formed was filtered, washed with water and air dried to yield the desired carboxylic acid.

MS (ES) C₁₄H₁₂N₂O₆S requires: 336, found: 337 (M+H⁺).

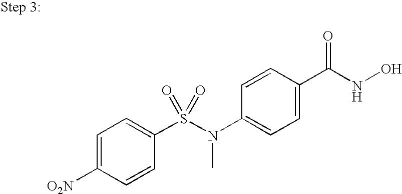

To a solution of the carboxylic acid from Step 2 above (0.033 g, 0.098 mMol) in CH₂Cl₂ (1.5 mL) was added O-t-butylhydroxylamine (0.050 g) at 0° C. followed by the addition of EDC (0.0.028 g, 0.146 mMol). The mixture was stirred at room temperature for 2 h and then diluted with CH₂Cl₂, washed with water, dried (Na₂SO₄) and concentrated under reduced pressure. The residue obtained was treated with TFA at room temperature overnight. The crude product obtained was purified by column chromatography on silica-gel using with CH₂Cl₂/MeOH (60:1) to yield the titled hydroxamic acid. ¹H NMR (400 MHz, d6-DMSO) δ 8.36 (d, 2H, J=7.0 Hz), 8.16 (d, 2H, J=7.0 Hz), 7.94 (d, 2H, J=7.0 Hz), 7.32 (d, 2H, J=7.0 Hz), 3.7 (3H, s). MS(ES) C₁₄H₁₃N₃O₆S requires: 351, found: 352 (M+H⁺). The following compounds of this invention described in Table 1 and 2 were prepared using the methods similar to that described in Examples 1 and 2, respectively.

TABLE 1

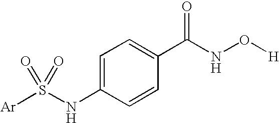

| | | Mass |
|---|---|---|
| Compound | | Spect: |
| # | Ar | (M + 1) |
| 1 | 4-Nitro-phenyl | 338 |
| 2 | 4-(N,N-Dimethylamino)phenyl | 336 |
| 3 | 4-t-Butyl-phenyl | 349 |
| 4 | 2-Carbomethoxy-phenyl | 351 |
| 5 | 2,5-Dimethoxy-phenyl | 353 |
| 6 | 3,4-Dimethoxy-phenyl | 353 |
| 7 | 4-Cyanophenyl | 318 |
| 8 | 3-(N,N-Dimethylamino)phenyl | 336 |
| 9 | 2-(N,N-Dimethylamino)phenyl | 336 |
| 10 | 4-Isopropyl-phenyl | 335 |
| 11 | 4-Methoxyl-phenyl | 323 |
| 12 | 3-Bromo-4,6-dimethoxyl-phenyl | 431 |
| 13 | Phenyl | 293 |
| 14 | 4-Carboxy-Phenyl | 337 |
| 15 | p-Toluyl | 307 |
| 16 | 4-(N,N-Dimethylcarboxamido)- | 364 |
| | phenyl | |
| 17 | 2-Nitro-4-trifluoromethyl-phenyl | 406 |
| 18 | Pentafluorophenyl | 383 |
| 19 | 4-Fluorophenyl | 311 |
| 20 | 2,5-Dichloro-phenyl | 361 |
| 21 | 3,5-Dichloro-6-hydroxy-phenyl | 377 |
| 22 | 2-Trifluoromethyl-phenyl | 361 |
| 23 | 2-Chloro-4-trifluoromethyl-phenyl | 395 |
| 24 | 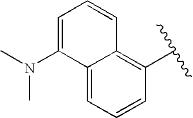 | 386 |
| 25 | 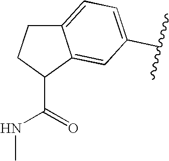 | 390 |
| 26 | 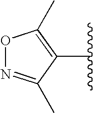 | 312 |
| 27 | 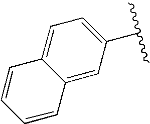 | 343 |
| 28 | 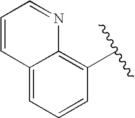 | 344 |
| 29 | 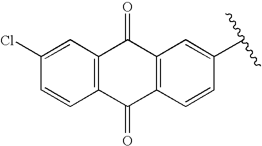 | 423 |
| 30 | 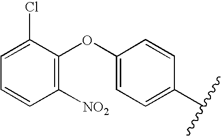 | 464 |
| 31 | 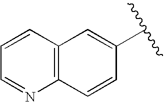 | 344 |
| 32 | 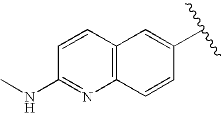 | 373 |
| 33 | 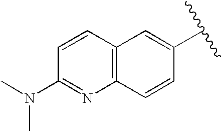 | 387 |
| 34 | 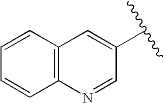 | 344 |
| 35 | 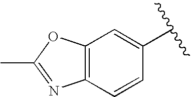 | 348 |
| 36 | 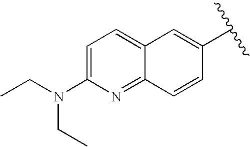 | 415 |
| 37 | 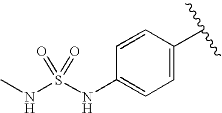 | 401 |
| 38 | 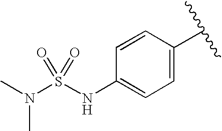 | 415 |
| 39 | 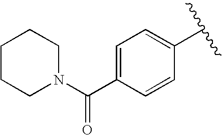 | 404 |
| 40 | 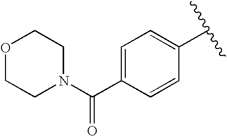 | 406 |
| 41 | 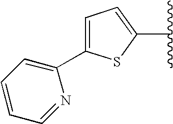 | 376 |
| 42 | 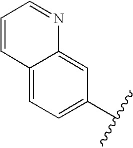 | 344 |
| 43 | 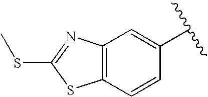 | 396 |
| 44 | 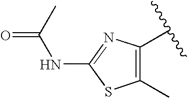 | 371 |
| 45 | 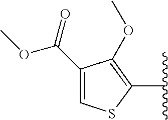 | 401 |
| 46 | 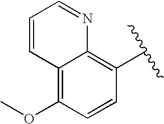 | 374 |
| 47 | 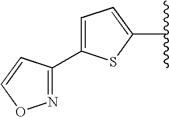 | 366 |
| 48 | 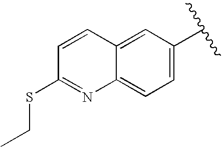 | 420 |
| 49 | 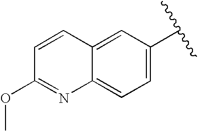 | 374 |
| 50 | 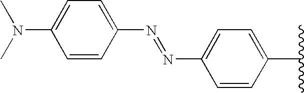 | 440 |
| 51 | 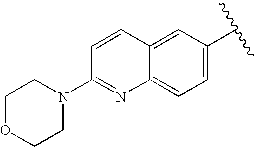 | 429 |
| 52 | 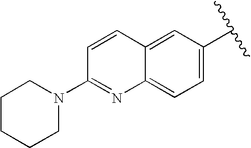 | 427 |
| 53 | 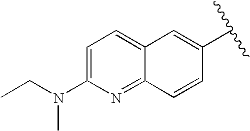 | 401 |
| 54 | 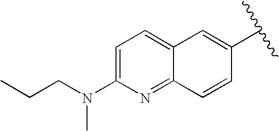 | 415 |
| 55 | 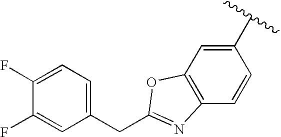 | 460 |
| 56 | 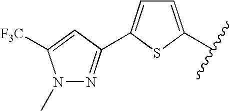 | 447 |
| 57 | 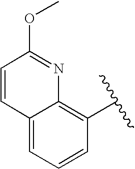 | 374 |
| 58 | 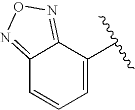 | 335 |
| 59 | 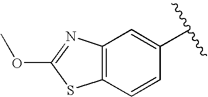 | 380 |
| 60 | 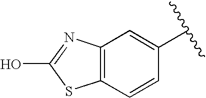 | 366 |
| 61 | 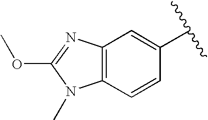 | 377 |
| 62 | 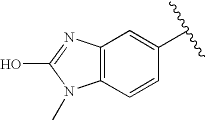 | 363 |
| 63 | 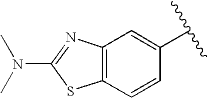 | 393 |
| 64 | 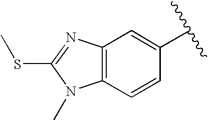 | 393 |
| 65 | 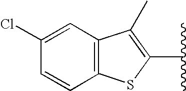 | 397 |
| 66 | 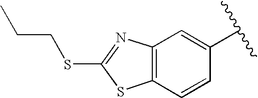 | 424 |
| 67 | 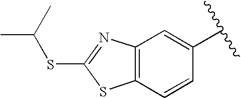 | 424 |
| 68 | 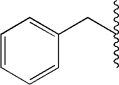 | 307 |
| 69 | 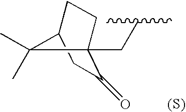 | 367 |
| 70 | 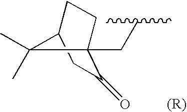 | 367 |

TABLE 2

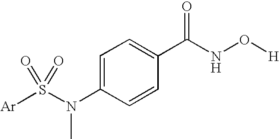

| | | Mass |
|---|---|---|
| Compound | | Spect: |
| # | Ar | (M + 1) |
| 71 | 4-Nitro-phenyl | 352 |
| 72 | 4-(N,N-Dimethylamino)phenyl | 350 |
| 73 | 4-t-Butyl-phenyl | 363 |
| 74 | 2-Carbomethoxy-phenyl | 365 |
| 75 | 2,5-Dimethoxy-phenyl | 367 |
| 76 | 3,4-Dimethoxy-phenyl | 367 |
| 77 | 4-Cyanophenyl | 332 |
| 78 | 3-(N,N-Dimethylamino)phenyl | 350 |
| 79 | 2-(N,N-Dimethylamino)phenyl | 350 |
| 80 | 4-Isopropyl-phenyl | 349 |
| 81 | 4-Methoxyl-phenyl | 337 |
| 82 | 3-Bromo-4,6-dimethoxyl-phenyl | 445 |
| 83 | Phenyl | 307 |
| 84 | 4-Carboxy-Phenyl | 351 |
| 85 | p-Toluyl | 321 |
| 86 | 4-(N,N-Dimethylcarboxamido)- | 378 |
| | phenyl | |
| 87 | 2-Nitro-4-trifluoromethyl-phenyl | 420 |
| 88 | Pentafluorophenyl | 397 |
| 89 | 4-Fluorophenyl | 325 |
| 90 | 2,5-Dichloro-phenyl | 375 |
| 91 | 3,5-Dichloro-6-hydroxy-phenyl | 391 |
| 92 | 2-Trifluoromethyl-phenyl | 375 |
| 93 | 2-Chloro-4-trifluoromethyl-phenyl | 409 |
| 94 | 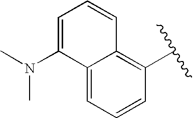 | 400 |
| 95 | 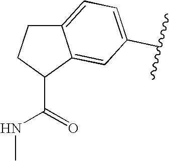 | 404 |
| 96 | 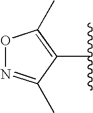 | 326 |
| 97 | 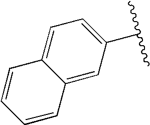 | 357 |
| 98 | 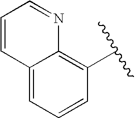 | 358 |
| 99 | 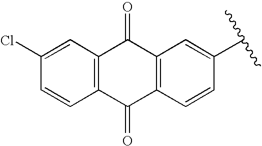 | 437 |
| 100 | 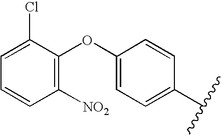 | 478 |
| 101 | 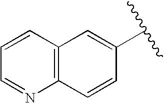 | 358 |
| 102 | 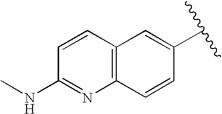 | 387 |
| 103 | 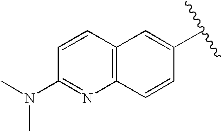 | 401 |
| 104 | 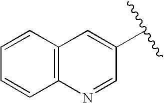 | 358 |
| 105 | 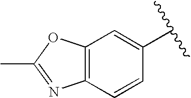 | 362 |
| 106 | 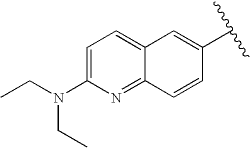 | 429 |
| 107 | 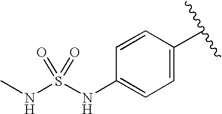 | 415 |
| 108 | 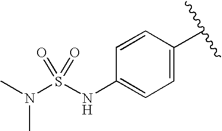 | 429 |
| 109 | 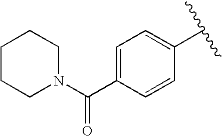 | 418 |
| 110 | 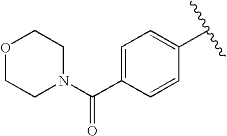 | 420 |
| 111 | 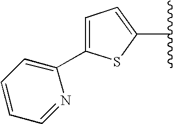 | 390 |
| 112 | 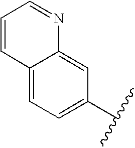 | 358 |
| 113 | 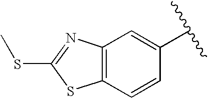 | 410 |
| 114 | 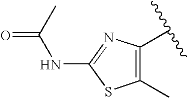 | 385 |
| 115 | 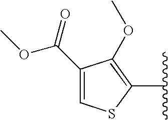 | 415 |
| 116 | 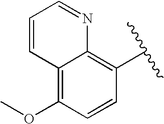 | 388 |
| 117 | 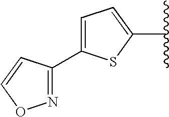 | 380 |
| 118 | 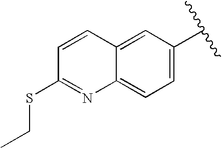 | 434 |
| 119 | 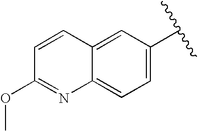 | 388 |
| 120 | 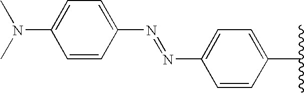 | 454 |
| 121 | 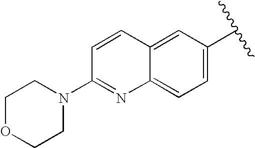 | 443 |
| 122 | 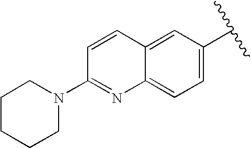 | 441 |
| 123 | 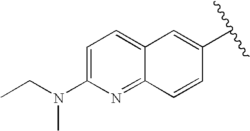 | 415 |
| 124 | 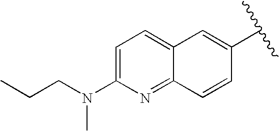 | 429 |
| 125 | 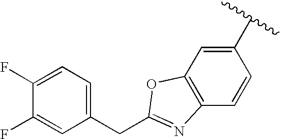 | 474 |
| 126 | 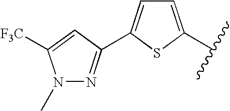 | 461 |
| 127 | 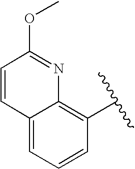 | 388 |
| 128 | 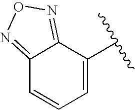 | 349 |
| 129 | 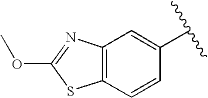 | 394 |
| 130 | 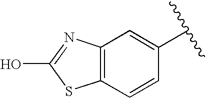 | 380 |
| 131 | 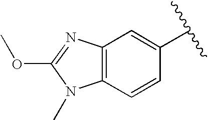 | 391 |
| 132 | 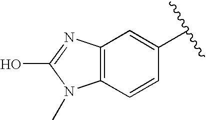 | 377 |
| 133 | 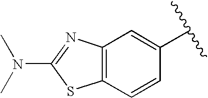 | 407 |
| 134 | 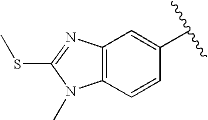 | 407 |
| 135 | 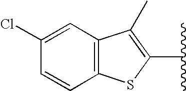 | 411 |
| 136 | 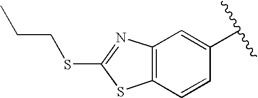 | 438 |
| 137 | 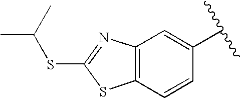 | 438 |
| 138 | 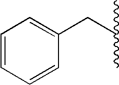 | 321 |
| 139 | 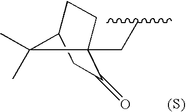 | 381 |
| 140 | 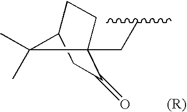 | 381 |

### EXAMPLE 3

### See Compound Number 141

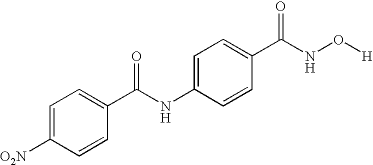

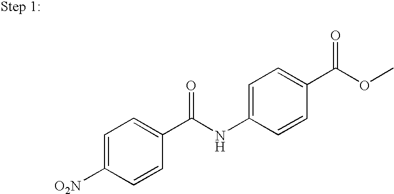

To a solution of 4-aminobenzoic methyl ester (1.26 g, 7 mMol) in CH₂Cl₂ and (40 mL) were added sequentially pyridine (1.4 mL), DMAP (0.1 g) and 4-nitrobenzoyl chloride (1.33 g, 7 mMol) at 0° C. The mixture was stirred at room temperature overnight. The precipitated product formed was filtered, washed with water and EtOAc and dried under suction to give the titled product. An additional 0.6 g of the product was isolated from the filtrate. MS(ES) C₁₅H₁₂N₂O₅ requires: 300, found: 301 (M+H⁺).

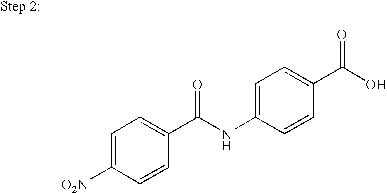

To a solution of the ester from Step 1 above (0.3 g, 1 mMol) in MeOH (6 mL) was added 2N NaOH (2 mL). The mixture was refluxed for 1 h and then the solvent was removed in vacuo. The residue obtained was dissolved in water (5 mL) and acidified with cold 1N HCl. The precipitate formed was filtered, washed with water and air dried to yield the desired carboxylic acid (0.2 g).

MS (ES) C₁₄H₁₀N₂O₅ requires: 286, found: 287 (M+H⁺).

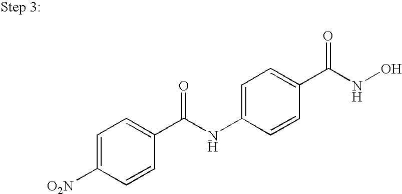

To a solution of the carboxylic acid from Step 2 above (0.1 g, 0.35 mMol) in a mixture of CH₂Cl₂ (3 mL) and DMF (0.5 mL) was added O-t-butyldimethylsilyl hydroxylamine (0.078 g, 0.53 mMol) at 0° C. followed by the addition of EDC (0.11 g, 0.575 mMol). The mixture was stirred at room temperature overnight. The reaction was diluted with CH₂Cl₂, washed with water, dried (Na₂SO₄) and concentrated under reduced pressure. The residue obtained was dissolved in a mixture of THF (2 mL), AcOH (1 mL) and water (1 mL) and stirred at room temperature overnight. The solvent was removed in vacuo and the crude product obtained was purified by column chromatography on silica-gel using with CH₂Cl₂/MeOH (60:1) to give the titled hydroxamic acid.

MS(ES) C₁₄H₁₁N₃O₅ requires: 301, found: 302 (M+H⁺).

The following compounds of this invention described in Tables 3 and 4 were prepared using the methods similar to that described in Example 3.

TABLE 3

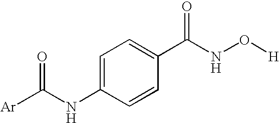

| | | Mass Spect: |
|---|---|---|
| Compound # | Ar | (M + 1) |
| 141 | 4-Nitro-phenyl | 302 |
| 142 | 4-(N,N-Dimethylamino)phenyl | 300 |
| 143 | 2-Carbomethoxy-phenyl | 315 |
| 144 | 3,4-Dimethoxy-phenyl | 317 |
| 145 | 4-Cyanophenyl | 282 |
| 146 | 3-(N,N-Dimethylamino)phenyl | 300 |
| 147 | 2-(N,N-Dimethylamino)phenyl | 300 |
| 148 | 4-Methoxyl-phenyl | 287 |
| 149 | Phenyl | 257 |
| 150 | 4-Carboxy-Phenyl | 301 |

TABLE 4

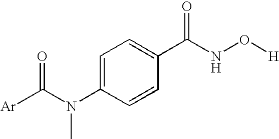

| | | Mass Spect: |
|---|---|---|
| Compound # | Ar | (M + 1) |
| 151 | 4-Nitro-phenyl | 316 |
| 152 | 4-(N,N-Dimethylamino)phenyl | 314 |
| 153 | 2-Carbomethoxy-phenyl | 329 |
| 154 | 3,4-Dimethoxy-phenyl | 331 |
| 155 | 4-Cyanophenyl | 296 |
| 156 | 3-(N,N-Dimethylamino)phenyl | 314 |
| 157 | 2-(N,N-Dimethylamino)phenyl | 314 |
| 158 | 4-Methoxyl-phenyl | 301 |
| 159 | Phenyl | 271 |
| 160 | 4-Carboxy-Phenyl | 315 |

The following list of compounds were made as the TFA salt: Compound nos. 2, 8-9, 24, 28, 31-34, 36, 41-42, 46, 48-49, 51, 53-54, 57, 72, 78-79, 94, 98, 101-104, 106, 111-112, 116, 118-119, 121-124, 127, 142, 146-147, 152 and 156-157.

### Assays

The compounds of the instant invention described in the Examples and shown in Tables 1-4 were tested in assays and were found to have HDAC inhibitory activity (IC₅₀ of ≦30 μM). Other assays are known in the literature and could be readily performed by those of skill in the art. Examples and protocols of assays useful for determining HDAC inhibitory acivity are found below.

HDAC Assay 1

Prepare 2.5 μl of compound or DMSO (20×) in 96 well microplate Packard Optiplate. To each well add 37.5 μl of Mix A, perform a 30 min. incubation at room temperature while shaking, then add 10 μl of Mix B, perform 3.5 hours incubation at room temperature while shaking, then add 10 μl of STOP Mix, incubate for 30 min. at room temperature and then read in FLUOSTAR ex355 nM em460/4 nM.

The final assay conditions contain: Hepes (pH 7.4, 50 mM), Glycerol (10%), BSA (0.1 mg/ml), Triton X100 (0.01%), Fluorogenic peptide IRBM91 (Boc-Ala-Ala-Lys[ε-Ac]-AMC; 20 uM), HeLa S3 extract from nuclei (20 μg/ml) or HDAC1 (1 nM), Lysyl End Peptidase (LEP; 0.25 mAu/ml) or Lysyl C endoprotease(LysC; 4.8 mU/ml) and Trichostatin A (1 μM).

The final assay volume is 50 μl.

Mix A contains: Buffer A 1× (37.5 μl), HeLa-S3 extract from nuclei (20 μg/ml; considering 50 μl/well) or HDAC1 (1 nM; considering 50 μl/well).

Mix B contains: Buffer A 1× (10 μl) and Pep IRBM91 (20 μM; considering 50 μl/well).

STOP Mix contains: Buffer A 1× (10 μl), LEP or Lys C (0.25 mAu/ml) or 4.8 mU/ml; considering 60 μl final volume) and Trichostatin A (1 μM; considering 60 μl final volume).

Buffer A 1× contains: Hepes (pH 7.4; 50 mM), Glycerol (10%), BSA (0.1 mg/ml) and Triton X100 (0.01%).

HDAC Assay 2

Prepare 2.5 μl of compound or DMSO (20×) in 96 well microplate Packard Optiplate. To each well add 37.5 μl of Mix A, then add 10 μl Mix B, incubate for 3.5 hours at room temperature while shaking, then add 25 μl SPA—Streptavidin beads (in buffer A 1×) and finally read in a Packard TOP COUNT.

The final assay conditions contain: Hepes (pH 7.4, 50 mM), Glycerol (10%), BSA (0.1 mg/ml), Triton X100 (0.01%), 3H Biotin-PEP439 (Biotin-G-A-[acetyl-3H]K—R—H—R-[acetyl-3H]K—V—NH₂, SPA-streptavidin beads (2 mg/ml) and HeLa S3 extract (40 μg/ml).

The final assay volume is 50 μl .

Mix A contains: Buffer A 2× (25 μl), HeLa-S3 extract (40 μg/ml) and H₂O (to 37.5 μl ).

Mix B contains: Buffer A 2× (5 μl), Pep 439 (50 nM; considering 50 μl final volume) and H₂O (to 10 μl).

Buffer A 2× contains: Hepes (pH 7.4; 100 mM), Glycerol (20%), BSA (0.2 mg/ml) and Triton X100 (0.02%).

Protocol for Nuclei Extraction from HeLa Cells (Adherent or in Suspension)

For a protocol on Nuclei extraction from HeLa S3 cells (adherent or in suspension) refer to Nare et al. 1999 Anal. Biochem., 267: 390-396.

Nuclei preparation for adherent HeLa S3 cells (0.5-1×109 cells) is as follows: wash cells twice with 1× PBS, scrape cells into 1× PBS, wash plates with 1× PBS, pool and spin cells at 800×g 10 minutes at 4° C., wash cell pellets with 1× PBS (count cells), spin cells at 800×g 10 minutes at 4° C., freeze cell pellets in liquid nitrogen and store −80° C.

Nuclei preparation for HeLa S3 cells in suspension (0.5-1×109 cells) is as follows: collect cells by centrifugation at 800×g 10 minutes at 4° C., wash cell pellets with 1× PBS, spin cells at 800×g 10 minutes at 4° C., repeat wash step twice (count cells), freeze cell pellet in liquid nitrogen and store at −80° C.

Resuspend cell pellets in lysis buffer (5 ml /1×108 cells; buffer contains: 0.25M sucrose, 0.45% NP40, 10 mM Tris-HCl (7.5), 10 mM NaCl, 5 mM MgCl_{2, 0.1} mM EGTA, 0.5 mM PMSF, COMPLETE protease inhibitor mix), vortex 10 sec and leave on ice for 15 minutes, spin through cushion (25 ml of lysate/5 ml cushion; cushion contains: 30% sucrose, 10 mM Tris-HCl (7.5), 10 mM NaCl, 3 mM MgCl₂), spin through cushion at 1,300×g 10 minutes at 4° C., remove super/cushion, resuspend in lysis buffer as above and re-spin through cushion as above, remove super/cushion.

For nuclear extraction, resuspend nuclear pellets in nuclei extraction buffer (13.5 ml/5 ml nuclear pellet; nuclei extraction buffer contains: 50 mM Hepes pH 7.4, (for use in HDAC ASSAY 2 also include 0.5 mM PMSF and COMPLETE protease inhibitor mix), sonicate into suspension on ice (1 min, output control between 4 and 5), leave on ice 30 min., centrifuge 100,000×g for 1 hr at 4° C., keep super on ice, repeat sonication/ice/centrifuge steps two more times, pool three supernatants and dialyze in 50 mM Hepes pH 7.4/10% glycerol and Snap-freeze suitable aliquots in liquid nitrogen and store −80° C.

Extraction and Purification Protocol for Flag-Tagged HDAC1 Expressed in HeLa Cells

HeLa cells transiently transfected with pCDNA3-HDAC1-FLAG are grown to 80% confluence on 10 cm culture dishes in DMEM, 10% Fetal bovine serum supplemented with antibiotics and glutamine. Cells are washed with 10 ml cold PBS and scraped into 2 ml of PBS. Cells are centrifuged for 5 minutes at 800×g at 4° C., washed with 30 ml PBS and resuspended in 10 ml PBS, counted, re-centrifuged and frozen at −80° C.

The frozen cell pellet is re-suspended in 1 ml of hypotonic lysis buffer (LB: 20 mM Hepes pH7.9, 0.25 mM EDTA, 10% glycerol) containing COMPLETE protease inhibitor and incubated on ice for 15 minutes, followed by homogenization on a 2-ml DounceB homogenizer (25 strokes). 150 mM KCl and 0.5% NP-40 are added to the homogenate and the solution is sonicated twice for 30 seconds (output5/6, duty cycle 90) and incubated for 1 hour at 4° C. After a 30 minutes centrifugation at 12000 rpm and 4° C. the supernatant (soluble extract) is collected and protein concentration is determined using the BIORAD assay.

Anti-FLAG M2 affinity resin (Sigma) is washed three times with TBS and twice with LB. 10 μl of the LB-washed resin/mg of protein (2-3 ug of Flagged-HDAC1) are added to the soluble extract (1 mL) and incubated overnight at 4° C. with gentle mixing. The resin is then collected by centrifugation, washed once with LB, twice with LB+0.1% NP40 and twice with elution buffer (50 mM Hepes pH 7.4, 5% glycerol, 100 mM KCl, 0.01% Triton X-100).

The affinity-purified HDAC is eluted from the resin by addition of a 10-fold excess (with respect to the resin) of elution buffer containing 100 μg/ml 3× FLAG peptide (SIGMA). The concentration of purified HDAC is determined by Western blot analysis.

What is claimed is:

1. A compound according to Formula I: 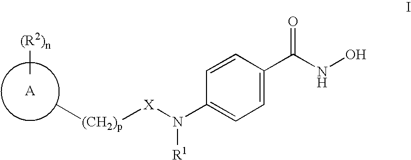
wherein:
a is 0 or 1; b is 0 or 1; m is 0, 1 or 2; n is 0, 1, 2, 3, 4 or 5; and p is 0, 1, 2 or 3; 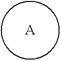
is cycloalkyl, aryl, heterocyclyl or 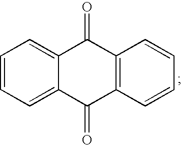
X is C═O or S(O)₂;
R¹ is selected from: H and (C₁-C₆)alkyl;
R² is independently selected from: oxo, OH, (C═O)ₐO_{b}(C₂-C₁₀)alkenyl, (C═O)ₐO_{b}(C₂-C₁₀)alkynyl, NO₂, (C═O)ₐO_{b}(C₁-C₆)alkyl, CN, (C═O)ₐO_{b}(C₃-C₁₀)cycloalkyl, halogen, (C═O)ₐ—N(R^{a})₂, CF₃, OH, NH—S(O)ₘ—R^{a}, (C═O)ₐO_{b}-heterocyclyl, (C═O)ₐO_{b}-aryl, S(O)ₘ—R^{a}, NH(C═O)R^{a}, N═N-aryl-N(R^{a})₂, (C₁-C₆)alkyl-aryl and heterocyclyl, said alkyl, alkenyl, alkynyl, cycloalkyl, aryl and heterocyclyl optionally substituted with one to three R^{b};
R^{a} is independently selected from: H and (C₁-C₆)alkyl;
R^{b} is independently selected from: oxo, NO₂, N(R^{a})₂, OH, CN, halogen, CF₃ and (C₁-C₆)alkyl;
or a pharmaceutically acceptable salt or stereoisomer thereof.

2. The compound according to claim 1 of the Formula I;
wherein: 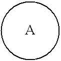
is phenyl, heterocyclyl or 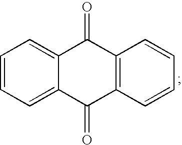
p is 0 or 1;
R¹ is CH₃;
and all substituents and variables are as defined in claim 1;
or a pharmaceutically acceptable salt or stereoisomer thereof.

3. The compound according to claim 2 of the Formula I; wherein:
R² is independently selected from: NO₂, (C═O)ₐO_{b}(C₁-C₆)alkyl, CN, (C₃-C₁₀)cycloalkyl, halogen, (C═O)ₐ—N(R^{a})₂, CF₃, OH, NH—S(O)ₘ—R^{a}, (C═O)ₐ-heterocyclyl, (C═O)ₐ-aryl, S(O)ₘ—R^{a}, NH(C═O)R^{a}, N═N-aryl-N(R^{a})₂, (C₁-C₆)alkyl-aryl and heterocyclyl, said alkyl, cycloalkyl, aryl and heterocyclyl optionally substituted with one to three R^{b};
R^{a} is independently selected from: H and (C₁-C₆)alkyl;
R^{b} is independently selected from: halogen, CF₃ and (C₁-C₆)alkyl;
and all substituents and variables are as defined in claim 2;
or a pharmaceutically acceptable salt or stereoisomer thereof.

4. A pharmaceutical composition comprising a pharmaceutical carrier, and dispersed therein, a therapeutically effective amount of a compound of claim 1.

5. The use of the compound according to claim 1 for the preparation of a medicament useful in the treatment or prevention of cancer in a mammal in need of such treatment.

6. The use of the compound according to claim 1 for the preparation of a medicament useful in the treatment or prevention of neurodegenerative diseses in a mammal in need of such treatment.

7. The use of the compound according to claim 1 for the preparation of a medicament useful in the treatment or prevention of schizophrenia in a mammal in need of such treatment.

8. The use of the compound according to claim 1 for the preparation of a medicament useful in the treatment or prevention of stroke in a mammal in need of such treatment.

9. The use of the compound according to claim 1 for the preparation of a medicament useful in the treatment or prevention of restenosis in a mammal in need of such treatment.

10. The use of the compound according to claim 1 for the preparation of a medicament useful in the treatment or prevention of protozoal infections in a mammal in need of such treatment.
